(19) United States
(12) Patent Application Publication
(10) Pub. No.: US 2008/0071380 A1
(43) Pub. Date: Mar. 20, 2008

(54) Systems and Methods for Percutaneous Placement of Interspinous Process Spacers

(76) Inventor: Thomas Sweeney, Sarasota, FL (US)

Correspondence Address: RADER, FISHMAN & GRAUER PLLC, 10653 SOUTH RIVER FRONT PARKWAY, SUITE 150, SOUTH JORDAN, UT, 84095, US

(21) Appl. No.: 11/857,260

(22) Filed: Sep. 18, 2007

Related U.S. Application Data
(60) Provisional application No. 60/845,686, filed on Sep. 19, 2006.

(51) Int. Cl. A61F 2/44

(52) U.S. Cl. 623/17.16

(57) ABSTRACT

An interspinous spacer configured to be implanted using minimally invasive techniques wherein the interspinous spacer is configured to deploy a first wing member on the distal side of an interspinous process and a second wing member on the proximal side of the interspinous process.

### RELATED APPLICATIONS

This application claims the benefit under 35 U.S.C. § 119(e) of U.S. Provisional Patent Application No. 60/845,686 filed Sep. 19, 2006 titled “Systems and Methods for Percutaneous Placement of Interspinous Process Spacers” which application is incorporated herein by reference in its entirety.

### BACKGROUND

The spinal column is a bio-mechanical structure composed primarily of ligaments, muscles, vertebrae and intervertebral disks. The bio-mechanical functions of the spine include the support of the body (which involves the transfer of the weight and the bending movements of the head, trunk, and arms to the pelvis and legs) and the protection of the spinal cord and the nerve roots.

As the present society ages, it is anticipated that there will be an increase in adverse spinal conditions which are characteristic of older people. By way of example, with aging comes increases in spinal stenosis (including but not limited to central canal and lateral stenosis), the thickening of the bones which make up the spinal column, and facet antropathy. Spinal stenosis is characterized by a reduction in the available space for the passage of blood vessels and nerves. Pain associated with such stenosis can be relieved by medication and/or surgery. Of course, it is desirable to eliminate the need for major surgery for all individuals and in particular for the elderly.

In addition, there are a variety of other ailments that can cause back pain in patients of all ages. For these ailments it is also desirable to eliminate such pain without major surgery. Accordingly, there needs to be eliminate such pain without major surgery. Accordingly, there needs to be developed implants for alleviating such conditions which are minimally invasive, can be tolerated by patients of all ages and in particular the elderly, and can be performed preferably on an outpatient basis.

### BRIEF DESCRIPTION OF THE DRAWINGS

The accompanying drawings illustrate various embodiments of the principles described herein and are a part of the specification. The illustrated embodiments are merely examples and do not limit the scope of the claims.

FIG. 1 is a side view of a portion of the spinal column, according to the principles described herein.

FIGS. 2A-2C are posterior views of an interspinous spacer design in various stages of deployment, according to one exemplary embodiment.

FIGS. 3A-3B are perspective views of an interspinous spacer design in various stages of deployment, according to one exemplary embodiment.

FIGS. 4A-4C are posterior views of an interspinous spacer design in various stages of deployment, according to one exemplary embodiment.

FIGS. 5A-5C are posterior views of an interspinous spacer in various stages of deployment, according to one exemplary embodiment.

FIGS. 6A-6C are posterior views of an interspinous spacer in various stages of deployment, according to one exemplary embodiment.

FIGS. 7A-7B are perspective views of an interspinous spacer design in various stages of engagement, according to one exemplary embodiment.

FIGS. 8A-8C are posterior views of an inflatable interspinous spacer design in various stages of deployment, according to one exemplary embodiment.

FIGS. 9A-9C are posterior views of a mechanically compliant interspinous spacer design in various stages of deployment, according to one exemplary embodiment.

FIG. 10 is a flow chart of an exemplary method of placing an interspinous spacer within the interspinous space, according to principles described herein.

Throughout the drawings, identical reference numbers designate similar, but not necessarily identical, elements.

### DETAILED DESCRIPTION

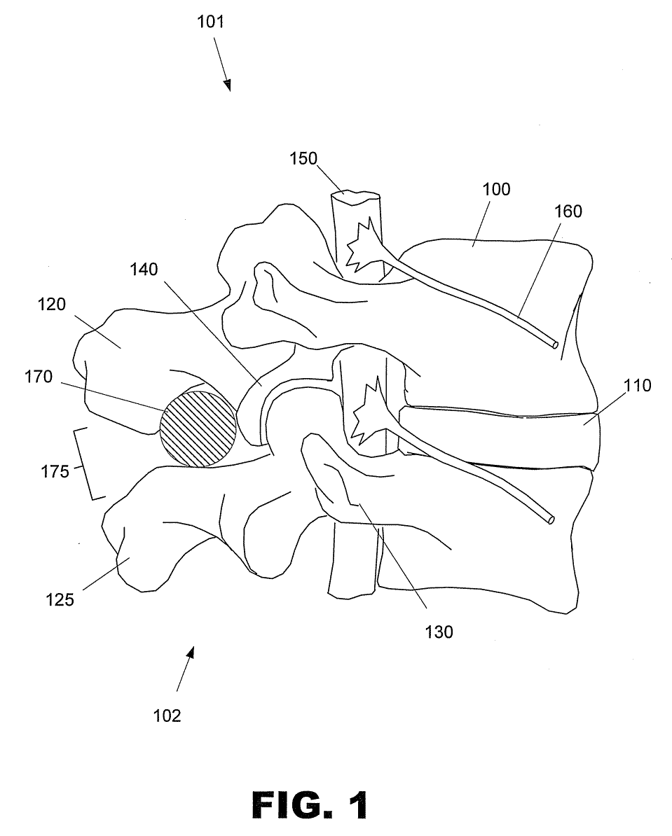

A number of exemplary interspinous spacer designs and methods for placing them are described herein. Particularly, a number of interspinous spacer designs that may be inserted into a patient using minimally invasive surgery techniques are disclosed herein. Various details of the designs will be provided below with reference to FIGS. 1A through 9C.

Before particular embodiments of the present system and method are disclosed and described, it is to be understood that the present system and method are not limited to the particular process and materials disclosed herein, as such may vary to some degree. It is also to be understood that the terminology used herein is used for the purpose of describing particular embodiments only and is not intended to be limiting, as the scope of the present system and method will be defined only by the appended claims and equivalents thereof.

In the following description, for purposes of explanation, numerous specific details are set forth in order to provide a thorough understanding of the present interspinous spacer designs. It will be apparent, however, to one skilled in the art, that the present systems and methods may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

FIG. 1 shows a side view of a segment of the spinal column comprising two adjoining vertebrae (101, 102). The vertebral column supports the body, which involves the transfer of the weight and the bending movements of the head, trunk, and arms to the pelvis and legs. The vertebral bodies (100) and intervertebral discs (110) support and transfer the weight of the body. The vertebral bodies (100) are dense, generally cylindrical bones with bony protrusions extending in the posterior and lateral directions. These protrusions include the transverse process (130) which extends a lateral direction from either side of the vertebral body (100) and the spinous process (140) which extends toward the posterior of the vertebral body (100). Intervertebral discs (110) are interposed between adjoining vertebral bodies (100) to provide spacing, cushioning, and flexibility to the vertebral column. The facet joints (140) limit and control the amount of motion between adjoining vertebral bodies (100). The spinal column is further supported by a plurality of muscles and ligaments (not shown) that surround the vertebrae.

The spinal column also provides protection for the spinal cord (150) and the nerve roots (160) that connect various parts of the body to the spinal cord (150).

The spine suffers from a variety of disorders arising from injury, age related degradation, hereditary influences, and others. When the spinal cord (150) or nerve roots (160) become compressed or pinched by the vertebrae, patients can suffer extreme and debilitating pain. By way of example and not limitation, one such disorder includes spinal stenosis. Spinal stenosis results from the thickening of bones that make up the spinal column and facet arthropathy. Spinal stenosis is characterized by a reduction in the available space for the passage of blood vessels and nerves.

In some cases, surgical intervention can mechanically decompress and stabilize the affected vertebrae through implanting supporting structures, thus relieving the pain and symptoms associated with compressed or pinched nerves. One such supporting structure is an interspinous spacer (170). The interspinous spacer (170) is placed between an upper spinous process (120) and a lower spinous process (125). The interspinous spacer (170) supports the contiguous vertebrae by limiting the backward bending motion of spinal column. By way of example and not limitation, an interspinous process spacer (170) can compensate for degradation of the facet joint (140) or the intervertebral disc (110), stabilize the vertebrae after spinal fusion, or limit the nerve compression caused by spinal stenosis.

However, spinal surgery can disturb and weaken the muscle and ligament structures that support the spine. To reduce the disruption of the surrounding tissues, minimally invasive surgical procedures can be used. Minimally invasive surgical procedures often involve the use of laparoscopic devices and remote manipulation of instruments through a small opening in the skin. Minimally invasive surgery can allow for outpatient surgical procedures, less pain and scarring, quicker recovery, and a lower incidence of post surgical complications.

Because the interspinous spacer (170) is placed between two dynamic bone structures (120,125), it can be helpful to provide additional retention features to prevent the spacer from becoming dislocated. For example, when the torso bends forward, such as when an individual bends over to pick up an item off the floor, the spine flexes, increasing the interspinous space (175). The dynamic nature of the interspinous space (175) creates additional demands on the design of an interspinous spacer (170). In some embodiments, a wing or flange greater than the diameter of the central portion of the interspinous spacer is provided on either side of the spacer (170) to prevent the spacer (170) from becoming dislocated.

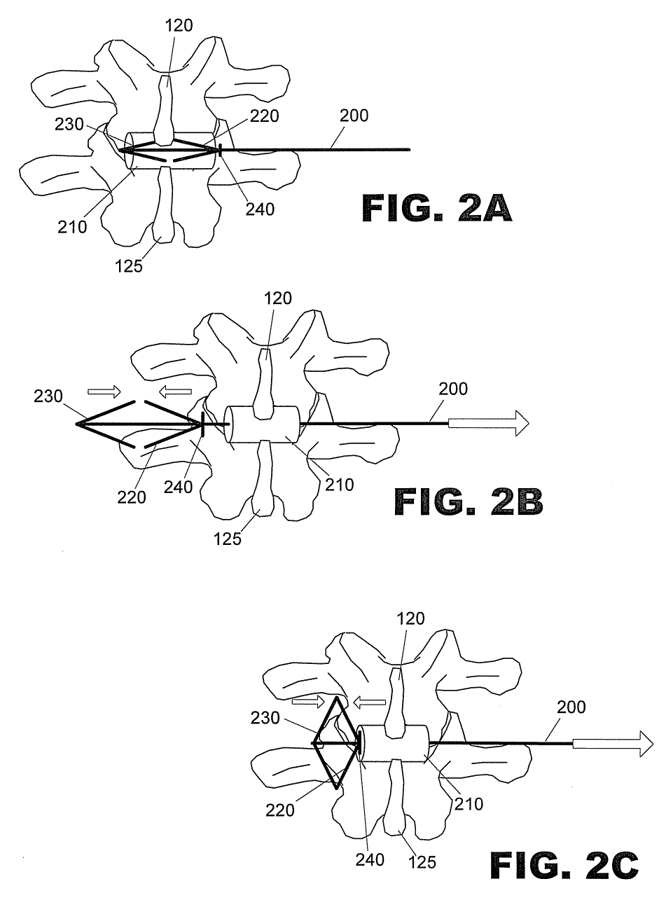

FIGS. 2A through 2C illustrate an interspinous spacer design, according to one exemplary embodiment. As illustrated in FIGS. 2A through 2C, the exemplary implant has a central cylinder (210) designed to pass through the interspinous area. As illustrated, a set of wings or flanges (220, 230) are disposed on one or both sides of the central cylinder (210). According to the present exemplary embodiment, the wings or flanges (220, 230) may be disposed inside or outside the central cylinder (210) in an un-deployed state during insertion.

As illustrated, once the central cylinder (210) is inserted in the interspinous area between an upper spinous process (120) and a lower spinous process (125), the wings or flanges (220, 230) are deployed to form a disk of a gradually increasing diameter. As illustrated in FIGS. 2B and 2C, the wings or flanges (220, 230) may initially be in an elongated position close to the central axis of the implant that is designed to be passed over a guide-wire (200) or other insertion tool. According to this exemplary embodiment, once the central cylinder (210) is properly passed through the interspinous ligament, a screw mechanism or other sliding mechanism can be used to bring the distal and proximal portions of the wings or flange together, thereby creating a disk of gradually increasing diameter. These integral “grommet-like” end pieces lock the implant into the interspinous space. Particularly, according to one exemplary embodiment illustrated in FIGS. 2B and 2C, a pulling of an actuating member, such as a wire or cord (200), may cause either a two piece wing or flange (220, 230), or a singular diamond cross-sectional shaped wing or flange to come into contact with a sliding stop (240). According to this exemplary embodiment, continued translation of the actuating member (200) (or advancement of a screw or other mechanism) will cause the distal end (230) and the proximal end (220) of the wing or flange to come closer together. As the proximal end (220) and distal end (230) join, the central portion of the wing or flange member will flare out, until it exceeds the diameter of the central cylinder, thereby restricting extraction or lateral movement of the central cylinder (210).

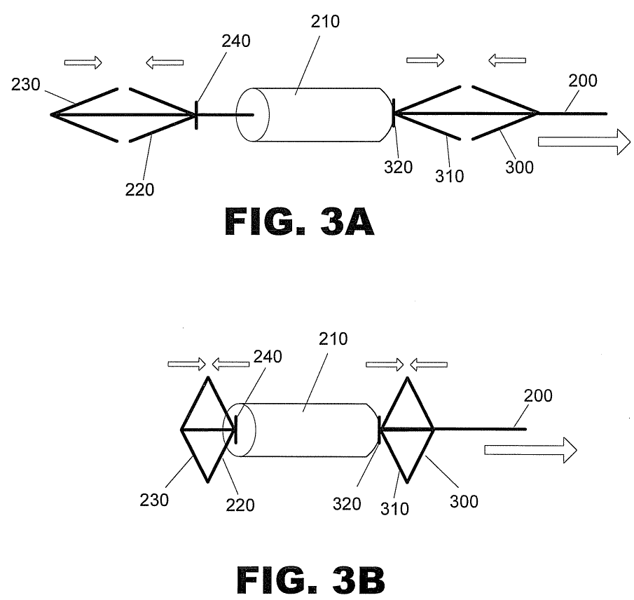

FIGS. 3A and 3B illustrate an alternative design. As illustrated, an additional actuating wing or flange member (300, 310) may be positioned opposite side of the central cylinder (210). Accordingly, when actuated, both of the actuating wings or flanges (220, 230; 300, 310) may be flared out to positionally fix the central cylinder (210) in the interspinous space (175, FIG. 1). Alternatively, a second wing or flange may be passed to one side of the central cylinder over a K-wire or other guide instrument during the procedure to fix the position of the second side of the central cylinder.

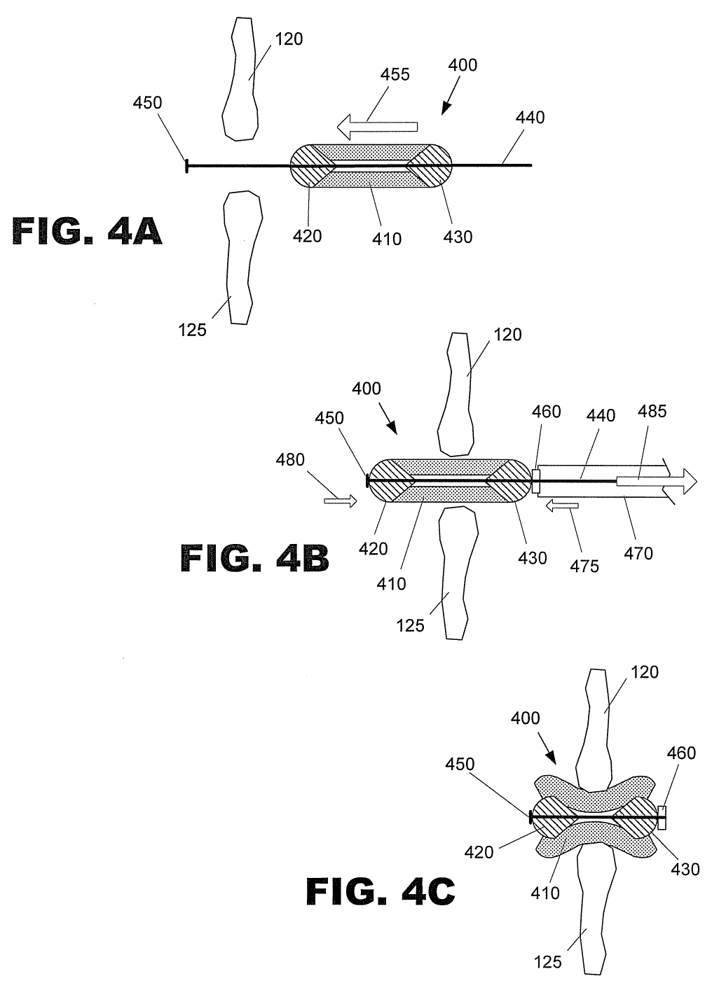

FIGS. 4A through 4C are cross-sectional diagrams that illustrate the geometry and method of inserting an alternative embodiment of the present invention into the interspinous space. FIG. 4A illustrates a K-wire or other probe (440) inserted between the upper spinous process (120) and the lower spinous process (125). In one embodiment, the probe has an integral stop (450). When the probe (440) has been placed in the desired interspinous location, a spacer (400) is passed over the probe (440) and moved toward the interspinous space as shown by the arrow (455). It is understood that a variety of additional surgical procedures could be performed prior to moving the spacer (400) into the interspinous space (175, FIG. 1). By way of example and not limitation, a series of trials may be inserted to expand the interspinous space or determine the correct sizing of the spacer. Additionally the initial probe or K-wire may be removed and a specialized probe (440) may be inserted.

FIG. 4B illustrates the spacer (400) positioned between the upper spinous process (120) and the lower spinous process (125). The spacer comprises a first body (420) and a second body (430) disposed at either end of a deformable element (410). The first and second bodies (420, 430) may assume a variety of shapes. In one exemplary embodiment, the bodies (420, 430) are conical with rounded exteriors. The deformable element may also assume a variety of geometries including, but not limited to, deformable elements with a cylindrical, rectangular, square, or elliptical transverse cross-sections.

Following the insertion of the spacer (400) into the desired interspinous location, the first and second bodies (420, 430) are brought together as indicated (475, 480). In one exemplary embodiment, a sliding stop (460) can be passed over the K-wire or specialized probe (440). The sliding stop (460) is translated toward the opposing stop (450) by means of a rigid instrument (470) that passes over the K-wire (440) and contacts the back of the sliding stop (460). The K-wire (440) is simultaneously retracted, as indicated by the arrow (485). The simultaneous translation of the sliding stop (460) and retraction of the K-wire (440) moves the first body (420) and the second body (430) toward each other.

As shown in FIG. 4C, the motion of the first body (420) and the second body (430) toward each other axially compresses and radially expands the deformable element (410). The deformable element (410) expands to fill the interspinous space, thereby providing a resilient support between the upper spinous process (120) and the lower spinous process (125). Additionally, the intrusion of the first and second bodies (420, 430) into the interior of the deformable element (410) provides integral wings on either side of the upper and lower interspinous bones (120, 125) which prevents the dislocation or shifting of the spacer (400).

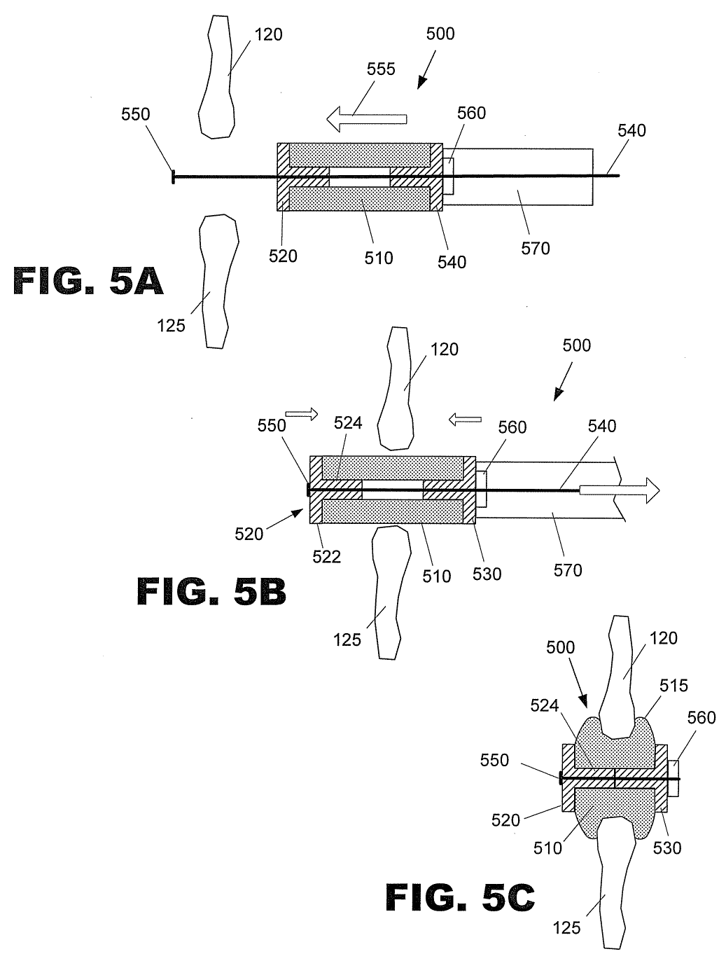

FIGS. 5A through 5C are cross-sectional diagrams that illustrate the geometry of an alternative embodiment of an interspinous spacer (500). Additionally FIGS. 5A through 5C illustrate the principles of operating and inserting the interspinous spacer (500). FIG. 5A illustrates a K-wire or other probe (540) inserted between the upper spinous process (120) and the lower spinous process (125). In one embodiment, the probe has an integral stop (550). When the probe (540) has been placed in the desired interspinous location, a spacer (500) is passed over the probe (540) and moved toward the interspinous space as shown by the arrow (555). As previously mentioned, a variety of additional surgical procedures could be performed in addition to those specifically discussed. The exemplary method is provided to illustrate the novel aspects of the invention and is not intended to be exhaustive. The surgical procedure may be varied according to the practice of the surgeon and the specific circumstances of the patient.

FIG. 5B illustrates the spacer (500) positioned in the interspinous space. The spacer (500) comprises a first body (520) and second body (530) disposed at either end of a deformable element (510). The first and second bodies (520, 530) may assume a variety of shapes. In one exemplary embodiment, the bodies (520, 530) consist of a cylindrical end piece (522) and a smaller diameter core piece (524). The deformable element (510) may also assume a variety of hollow geometries including, but not limited to, objects with a cylindrical, rectangular, square, or elliptical transverse cross-sections.

Following the insertion of the spacer (500) into the desired interspinous location, the first and second bodies (520, 530) can be brought together in a fashion similar to that illustrated in FIGS. 4A through 4C. In one exemplary embodiment, a sliding stop (560) can be passed over the K-wire or specialized probe (540). The sliding stop (560) is translated toward opposing stop (550) by means of a rigid instrument (570) that passes over the K-wire (540) and contacts the back of the sliding stop (560). The K-wire (540) is simultaneously retracted, resulting in the motion of the first and second bodies (520, 530) toward each other and the compression of the deformable element (510).

Other methods of bringing the first and second bodies (420, 430; 520, 530) together could be used as well. By way of example and not limitation, the probe (540) or another interior element could be threaded. The sliding stop (460, 560) could consist of a nut configured to receive the threaded element. The nut could be rotated about the threaded element, thereby bringing the first and second bodies together.

As shown in FIG. 5C, the axial compression of the deformable element (510) causes the deformable element (510) to fill the interspinous space (175, FIG. 1), thereby providing resilient support between the upper spinous process (120) and the lower spinous process (125). In one exemplary embodiment, the first and second bodies (520, 530) could be brought together such that the core pieces (524) meet in the center of the spacer. This provides a solid core surrounded by the deformable element (510). In one exemplary embodiment, the deformable element (510) expands and conforms to the outer surfaces of the upper spinous process (120) and the lower spinous process (125) such that the deformable element (510) forms wings or flanges (515) on either side of the bones (120, 125) that conform to the shape of the bones to prevent the dislocation or shifting of the spacer (500).

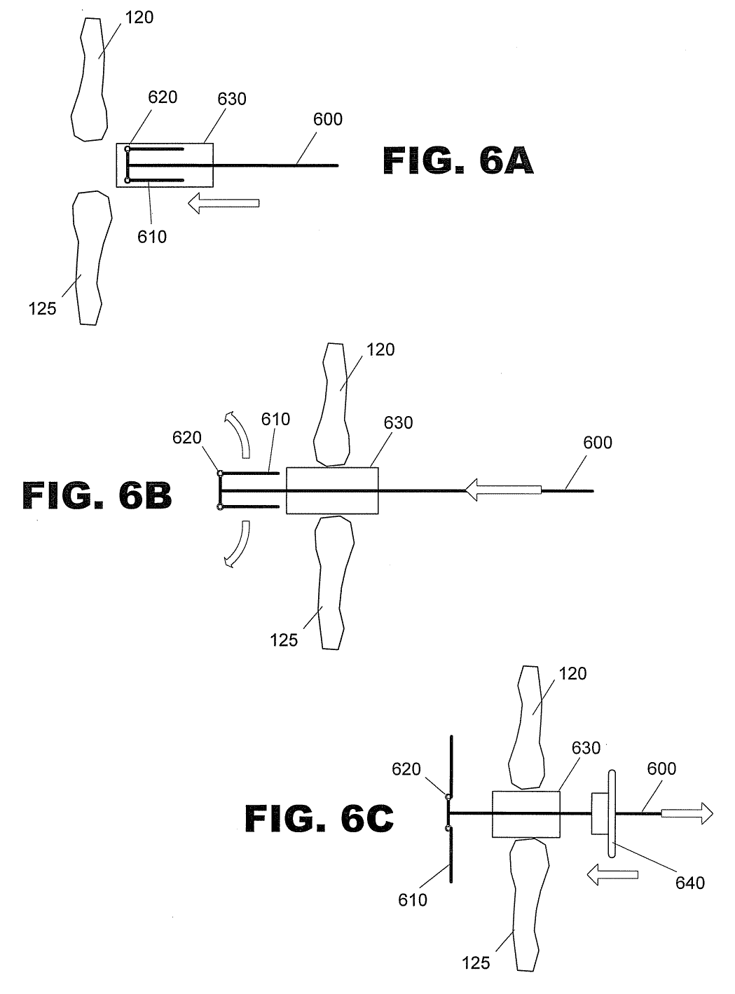

FIGS. 6A through 6C illustrate another exemplary method for deploying a wing or flange on either side of a central cylinder. As shown in FIGS. 6A and 6C, the wing or flange member (610) may be a spring loaded member held in a stowed configuration within the central cylinder (630). According to this exemplary embodiment, the walls of the central cylinder (630) resist the spring force exerted by the spring hinges (620). However, when released from within the central cylinder (630) as shown in FIG. 6B, the spring hinges (620) deploy and extend the flange members. The flange members (610) assist in positionally fixing the central cylinder (630) within the interspinous space (175, FIG. 1). While the exemplary embodiment illustrated in FIGS. 6A through 6C are shown with spring hinges (620), the spring motion may be provided by the compliant properties of the material forming the wing or flange member (610).

FIG. 6C shows the deployed wings (610) being translated back toward the spinous process bones (120, 125) by a force exerted along the insertion support (600). Additionally, a flange (640) can be passed over the insertion support (600) to form the opposing wing, thereby stabilizing the position of the cylinder (630) within the interspinous space.

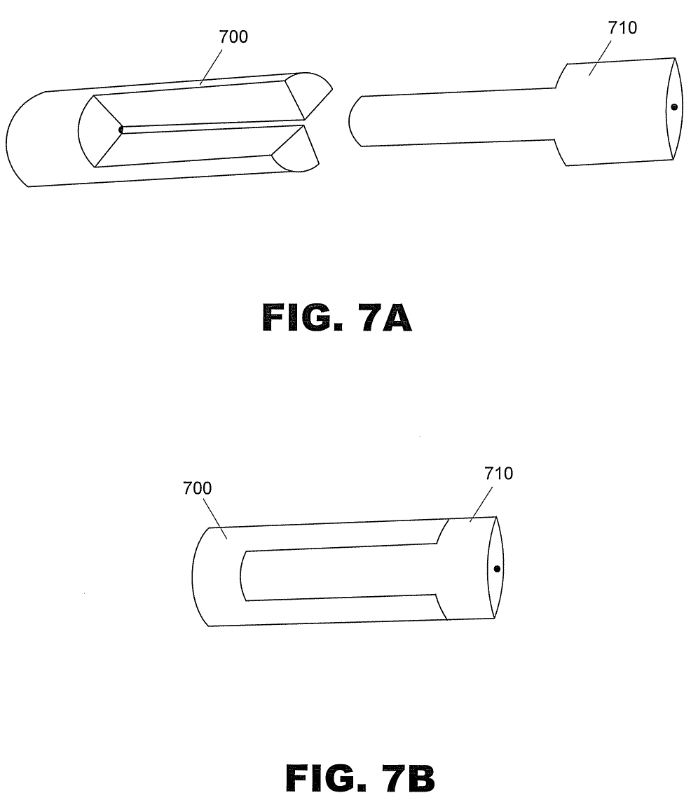

In addition, rather than using a hollow central cylinder, any number of cylindrical implants may be used to be placed within the interspinous space. As illustrated in FIGS. 7A and 7B, a two-part implant may be inserted in the interspinous space. Particularly, after tensioning the interspinous space with a series of trials, a two-part implant can be inserted. According to this exemplary embodiment, a first portion (700) of the two-part implant is placed over the guide-wire (not shown). Once the first flattened component (700) is placed, the second component (710) slides over and mates with the first component (700) such that a cylinder is formed within the interspinous space, as illustrated in FIG. 7B. Once the two mating portions (700, 710) of the two-part implant are positioned, the end wings, grommets, or flanges can then be deployed using various mechanisms.

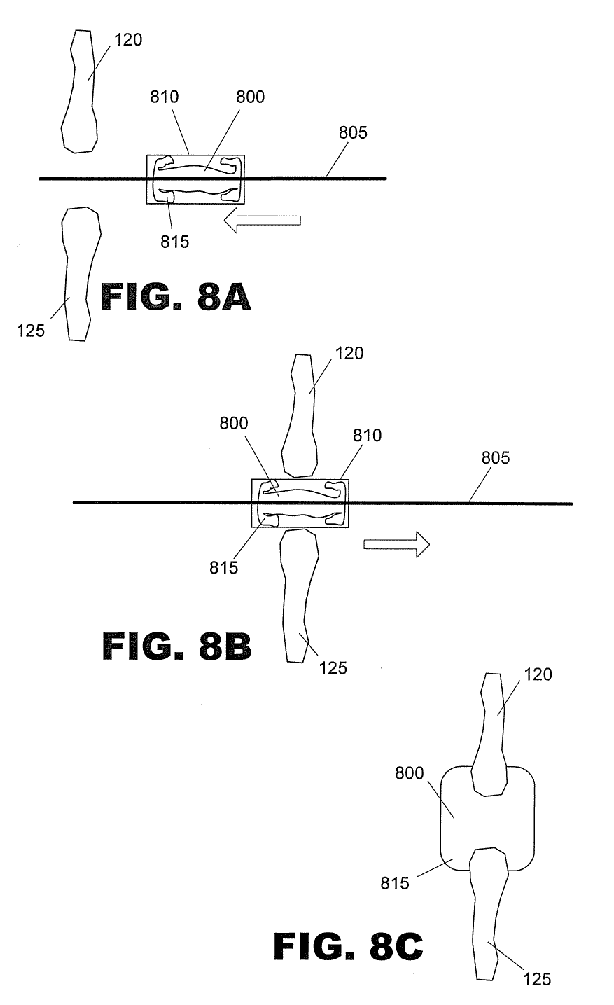

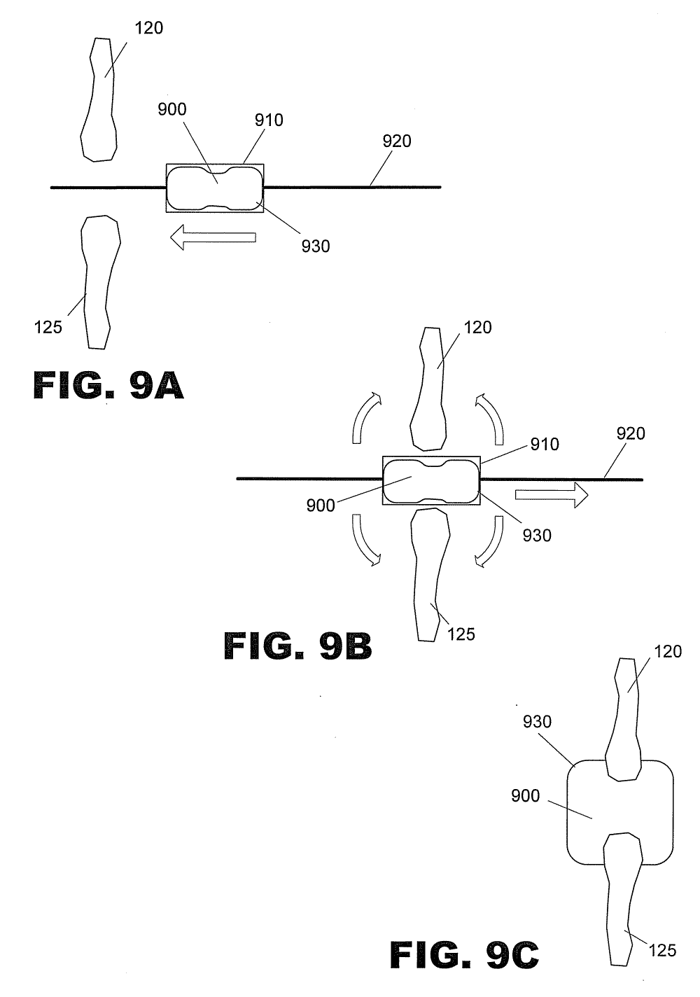

Furthermore, a non-solid member may be used to form the interspinous implant, as illustrated in FIGS. 8A through 8C and FIGS. 9A through 9C. FIGS. 8A through 8C show one exemplary embodiment in which an expandable bladder or balloon (800) which may be disposed, in a deflated state, within a central cylinder (810). According to this exemplary embodiment, the bladder or balloon (800) is coupled to an inflation tube (not shown) which can pass through the skin. The bladder (800) is intraoperatively inflated to varying degrees to match distraction determined by trials. Additionally, as illustrated, the insertion and inflation of the bladder is performed in a percutaneous or minimally invasive manner. The balloon tube (not shown) can be left passing through the skin so that postoperatively, as an outpatient or inpatient, the pressure within the balloon be altered to adjust the distraction between the interspinous processes. Additionally, an assessment can be made on how various pressures within the balloon affect the patient's symptoms. When optimal inflation of the balloon or bladder is determined, then using the exiting tube, a crimping mechanism is deployed or valve mechanism is used to prevent expulsion of the fluid contained within the balloon. According to one exemplary embodiment, the tube itself can be left in a subcutaneous location or the tube can be detached from the balloon.

According to one exemplary embodiment, the bladder or balloon may be a dumbbell shaped inflatable device which can be placed over a guide-wire (805) or through a tubular access port into the interspinous space (175, FIG. 1). Once in place, the variable pressure can be applied to the fluid within the balloon (800) to alter the distraction between the interspinous processes (120, 125). Once optimum amount of distraction is determined, the inflating tube can be closed, detached, or left in a subcutaneous position. This device can be modified as an outpatient; and if no desired effect is achieved, it could be deflated and easily removed in an outpatient or office setting.

As shown in FIGS. 8A through 8C, the bladder or balloon (800) may be placed within a tube (810). Once within the interspinous space (175, FIG. 1), the tube (810) may be removed and the bladder or balloon (800) inflated. As shown, the balloon or bladder (800) may be formed to include retention wings or flanges (815), thereby providing for positional maintenance.

In yet another alternative embodiment, illustrated in FIGS. 9A through 9C, an interspinous implant may be formed of a single compliant material. Particularly, according to one exemplary embodiment, the interspinous implant can be a silicone dumbbell-shaped or grommet-shaped spacer (900) which is again cannulated in a similar fashion to the above noted designs. According to this exemplary embodiment, once the guide-wire (920) is appropriately positioned, trials can be placed to expand the interspinous space. The grommet (900) can then be deployed along the guide wire (920) to the desired interspinous region. Once the grommet (900) is in position, the cylinder (910) can be withdrawn with counter pressure, keeping the grommet (900) in the desired interspinous space. Following the removal of the cylinder (910) the grommet (900) expands to fill a portion of the interspinous space. The grommet phalanges or wings (930) are deployed on right and left sides of the interspinous ligament as shown in FIG. 9C.

### Exemplary Placement Method

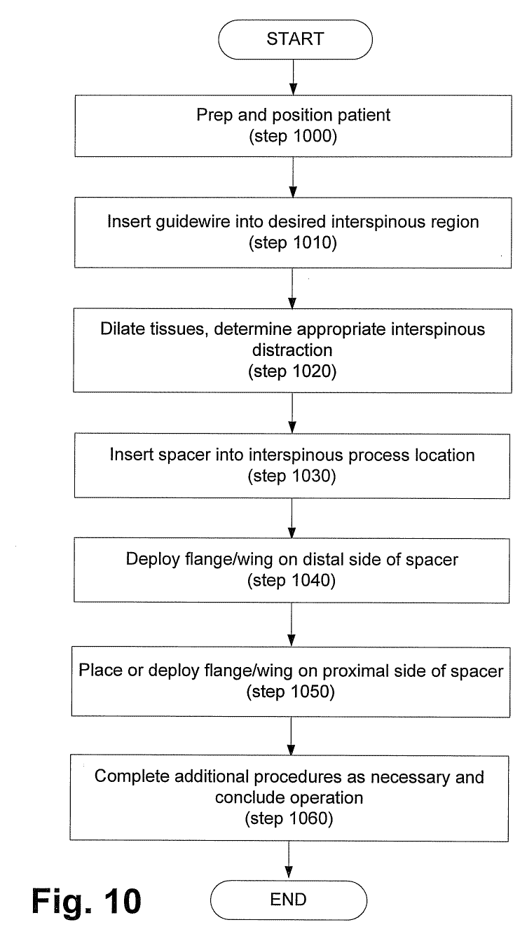

While any number of methods may be used for placing the present exemplary interspinous process spacers in appropriate locations of the lumbar, thoracic, or cervical spine of a patient, an exemplary method will be provided herein. The exemplary method is diagrammed in FIG. 10 as a flow chart. With the patient prepped and draped using local regional or general anesthetic, he/she is positioned in the lateral position right or left (step 1000). A guide-wire is then used to approach the interspinous space perpendicular to the sagittal plane starting from the top side where the patient is positioned. The guide-wire is then passed through the skin and through the interspinous ligament in a position between the facet joints and the supra-spinous ligament (step 1010). Having performed these preliminary procedures, a minimal “stab” incision is made about the guide-wire to allow passage of the implant. First a series of trials are used to pass through the interspinous space, assessing tension and distraction of the space (step 1020). After the appropriate distraction is determined, an implant is chosen, and the implant is then passed over the guide-wire percutaneously through the interspinous ligament (step 1030).

The mechanism of the distal flange or wings is deployed (step 1040), and then the mechanism of the proximal flange or wings is deployed (step 1050). This technique is applicable to implants that are delivered without separate parts or implants that are delivered with deployable distal and mid portions. Following the deployment of the distal flange or wing, a proximal flangeal wing may be slid down the guide-wire and attaching to the implant. Alternatively, an implant can be passed over the guide-wire percutaneously which includes the proximal wing or flange and the central portion which passes through the interspinous ligament and then through a separate percutaneous approach on the opposite side. The wing or phalange can be placed over the guide-wire or freehand to complete construction of the interspinous spacer. For embodiments of spacers that simultaneously deploy both the distal and proximal flanges, such as the spacers illustrated FIGS. 4A-4C, 5A-5C, 8A-8C, 9A-9C, steps 1040 and 1050 may be merged.

Following the deployment of the flanges, additional procedures can be performed as required and the operation can be concluded (step 1060). The additional procedures can, by way of example and not limitation, include withdrawal of the K-wire, securing locking mechanisms such as sliding stops (320, 460, 560), withdrawing insertion aids such as cylinders (810, 910), disconnecting or severing wires of which a portion remains inside the spacer (440, 540, 600), and any other required tasks.

In conclusion, the present exemplary systems and methods provide for the insertion of an interspinous spacer using minimally invasive surgical techniques. Particularly, as mentioned above, a number of implant designs are disclosed that provide for the interoperative deployment of flanges or wings to maintain the position of an interspinous spacer.

The preceding description has been presented only to illustrate and describe exemplary embodiments of the present system and method. It is not intended to be exhaustive or to limit the system and method to any precise form disclosed. Many modifications and variations are possible in light of the above teaching. It is intended that the scope of the system and method be defined by the following claims.

What is claimed is:

1. An interspinous spacer configured to be placed and deployed using minimally invasive techniques, comprising:
a spacer member; and
at least one wing member associated with said spacer.

2. The interspinous spacer of claim 1 wherein said at least one wing member consists of a distal wing member and a proximal wing member.

3. The interspinous spacer of claim 2 wherein said spacer member extends along a lateral axis between said distal wing member and said proximal wing member; and said distal wing member and said proximal wing member are configured to be deployed parallel to a longitudinal axis, said longitudinal axis being generally perpendicular to said lateral axis.

4. The interspinous spacer of claim 3 wherein said interspinous spacer is configured to be deployed along a K-wire or other probe.

5. The interspinous spacer of claim 4 wherein said distal wing member is configured to be remotely deployed on a distal side of an interspinous space without open surgical access said distal side of said interspinous space.

6. The interspinous spacer of claim 1, wherein said spacer member comprises a first implant member and a second implant member; wherein said first implant member and said second implant member are configured to be slideably joined to form said spacer member.

7. The interspinous spacer of claim 6, wherein said first implant member and said second implant member are substantially identical.

8. The interspinous spacer of claim 1, wherein said at least one wing member comprises an expandable wing member, said expandable wing member additionally comprises:
a first conical member; and
a second conical member;
wherein said first conical member and said second conical member are configured to be joined and expanded to form a disk of increasing diameter.

9. The interspinous spacer of claim 8, wherein said expandable wing member comprises a diamond shaped member configured to be expanded as said diamond shaped member is compressed.

10. The interspinous spacer of claim 1, wherein said interspinous spacer comprises:
a substantially horizontal member;
a substantially vertical member coupled to said substantially horizontal member in a perpendicular orientation; and
at least one spring loaded hinge member formed on at least one end of said substantially horizontal member.

11. The interspinous spacer of claim 10, further comprising a grommet configured to be coupled to a second end of said spacer.

12. The interspinous spacer of claim 5, wherein said spacer member and said at least one wing member comprise at least one bladder or balloon member.

13. The interspinous spacer of claim 5, wherein said spacer member and said at least one wing member comprise a single compliant material formed in a dumbbell like shape.

14. The interspinous spacer of claim 13, wherein said compliant material comprises silicone.

15. An interspinous spacer configured to be inserted into an interspinous space through minimally invasive surgical techniques comprising:
a first body disposed on a first end of said interspinous spacer;
a second body disposed on a second end of said interspinous spacer;
a deformable element interposed between said first body and said second body.

16. The interspinous spacer of claim 15 wherein said interspinous spacer is configured to be inserted into said interspinous space by passing said interspinous spacer over a central member.

17. The interspinous spacer of claim 16 wherein said interspinous spacer is configured such that when said first body and said second body are moved axially toward each other, said deformable element is axially compressed and radially expanded to contact a first spinous process and a second spinous process.

18. The interspinous spacer of claim 17 wherein said expanded deformable element extends around said first spinous process and said second spinous process to secure said interspinous spacer.

19. A method for inserting an interspinous spacer comprising:
inserting a guide-wire into an interspinous space perpendicular to a sagittal plane;
making a minimal “stab” incision about said guide-wire;
passing a series of trials through said interspinous space, assessing tension and distraction of said interspinous space; and
passing a interspinous spacer over said guide-wire percutaneously through the interspinous ligament.

20. The method of claim 19 further comprising the steps of
deploying a distal flange of said interspinous spacer;
sliding a proximal flangeal wing down said guide-wire; and
attaching said proximal flangeal wing to said interspinous spacer.
